(19) United States
(12) Patent Application Publication
(10) Pub. No.: US 2019/0139454 A1
(43) Pub. Date: May 9, 2019

(54) OPERATION TRAINING APPARATUS

(71) Applicants: TOYODA GOSEI CO., LTD., Kiyosu-shi (JP); EBM CORPORATION, Tokyo (JP)

(72) Inventors: Takeshi Fujiwara, Kiyosu-shi (JP); Masatoshi Shimada, Kiyosu-shi (JP); Sara Kato, Kiyosu-shi (JP); Daisuke Matsuda, Kiyosu-shi (JP); Youngkwang Park, Tokyo (JP)

(21) Appl. No.: 16/176,205

(22) Filed: Oct. 31, 2018

(30) Foreign Application Priority Data
Nov. 7, 2017 (JP) 2017-214424
Jun. 28, 2018 (JP) 2018-123642
Jun. 28, 2018 (JP) 2018-123643

(51) Int. Cl. G09B 23/32; A61B 17/00; G09B 23/30

(52) U.S. Cl. CPC G09B 23/32; A61B 2017/00716; G09B 23/303; A61B 17/00

(57) ABSTRACT

An operation training apparatus is used in training for operations on living tissue that expands and contracts. The apparatus includes a model body, which mimics the living tissue, a driving device, which moves the model body in a manner mimicking the expansion-contraction, and a controller, which controls a manner in which the driving device is driven. The driving device includes an electroactive polymer actuator.

### BACKGROUND

The present disclosure relates to an operation training apparatus.

Japanese Laid-Open Patent Publication No. 2009-122130 discloses a surgical operation training apparatus used in training for beating-heart coronary artery bypass surgery.

The surgical operation training apparatus includes a model body, a holding body, a support body, wires, and a control unit. The model body includes a blood vessel model and a myocardium model. The holding body holds the model body. The support body operably supports the holding body. The wires join the holding body and the support body to each other. The control unit controls the operation of the holding body. The holding body includes a holding plate, a cylindrical central projecting portion, a coil spring, and multiple cylindrical corner projecting portions. The holding plate is attached to the lower surface of the myocardium model. The central projecting portion projects downward from a central section of the lower surface of the holding plate. The coil spring is attached to the central projecting portion. The corner projecting portions each project downward from one of the corner portions of the lower surface of the holding plate. The wires are made of thermally contractible Ti—Ni or Ti—Ni—Cu shape memory alloy and join the corner projecting portions to the support body. The control unit changes the supply state of an electric current supplied to the wires, thus changing the shape of the wires and correspondingly controlling the operation of the holding body.

The above-described conventional surgical operation training apparatus is capable of moving the model body, which is held by the holding body, at 60 to 100 beats per minute (BPM). This BPM corresponds to a typical adult resting heart rate.

Compared to the typical adult's case, the child's heart rate is as high as, for example, approximately 150 BPM. This makes operations on children highly difficult, thus increasing the necessity for training such operations. However, as described in the aforementioned document, the surgical operation training apparatus employs wires made of shape memory alloy as an actuator for operating the holding body. This prolongs the time from when the electric power supply is started to when the temperature of the wires rises or from when the electric power supply is interrupted to when the temperature of the wires drops. That is, the holding body operates at a low operating speed, in other words, the response of the holding body is slow. As a result, the above-described surgical operation training apparatus is not adapted for training for operations on models of high-heart-rate patients, such as children.

The above-described problem is not limited to a training apparatus for beating-heart coronary artery bypass surgery. The problem also occurs in training apparatuses for operations on other types of living tissue that expands and contracts or operations on pulsating blood vessels.

Also, in the surgical operation training apparatus described in the aforementioned document, the model body is held by the holding plate, which is a component of the holding body, and the holding plate is elastically supported by the coil spring. Therefore, even when a section of the model body is pressed by forceps, for example, the model body does not move easily in the pressing direction and tends to move in an unnatural manner. The surgical operation training apparatus thus needs to be more improved in terms of mimicking the heart.

The above-described problem is not restricted to training apparatuses for beating-heart coronary artery bypass surgery, but also occurs also in training apparatuses for operations on other types of living tissue.

### SUMMARY

A first objective of the present disclosure is to provide an operation training apparatus that can be adapted for training for a wide variety of operations.

A second objective of the disclosure is to provide an operation training apparatus having a model body that moves in a manner imitating the actual movement of living tissue.

To achieve the foregoing first objective, a first aspect of the present disclosure provides an operation training apparatus used in training for operation on living tissue that expands and contracts. The apparatus includes a model body, which mimics the living tissue, a driving device, which moves the model body in a manner mimicking the expansion-contraction, and a controller, which controls a manner in which the driving device is driven. The driving device includes an electroactive polymer actuator.

To achieve the foregoing first objective, a second aspect of the present disclosure provides an operation training apparatus used in training for operations on a pulsating blood vessel. The apparatus includes a blood vessel model, which mimics the blood vessel, a driving device, which moves the blood vessel model in a manner mimicking pulsation of the blood vessel, and a controller, which controls a manner in which the driving device is driven. The driving device includes an electroactive polymer actuator.

To achieve the foregoing second objective, a third aspect of the present disclosure provides an operation training apparatus used in training for operations on a living tissue. The operation training apparatus includes a model body that mimics the living tissue and a soft membrane that supports the model body.

Other aspects and advantages of the present disclosure will become apparent from the following description, taken in conjunction with the accompanying drawings, illustrating exemplary embodiments.

### BRIEF DESCRIPTION OF THE DRAWINGS

The disclosure may be understood by reference to the following description together with the accompanying drawings:

FIG. 1 is a side view showing a first embodiment of an operation training apparatus;

FIG. 2 is a cross-sectional view taken along line 2-2 of FIG. 1, representing the relationship among the positions of the driving devices of the first embodiment;

FIG. 3 is a perspective view showing a model body of the first embodiment;

FIG. 4 is a cross-sectional view showing one of the driving devices of the first embodiment;

FIG. 5 is an enlarged cross-sectional view showing a dielectric actuator as a component of a driving device of the first embodiment;

FIG. 6 is an electric circuit diagram representing the dielectric actuator of the first embodiment;

FIG. 7 is a view schematically showing the driving devices and a controller of the first embodiment;

FIG. 8 is a perspective view showing a model body of a second embodiment;

FIG. 9 is a cross-sectional view taken along line 9-9 of FIG. 8;

FIG. 10 is a perspective view showing an operation training apparatus of a third embodiment;

FIG. 11 is a cross-sectional view taken along line 11-11 of FIG. 10;

FIG. 12 is a plan view showing a dielectric actuator of the third embodiment;

FIG. 13 is a cross-sectional view taken along line 13-13 of FIG. 12; and

FIG. 14 is a view schematically showing the dielectric actuator and a controller of the third embodiment.

### DETAILED DESCRIPTION

### First Embodiment

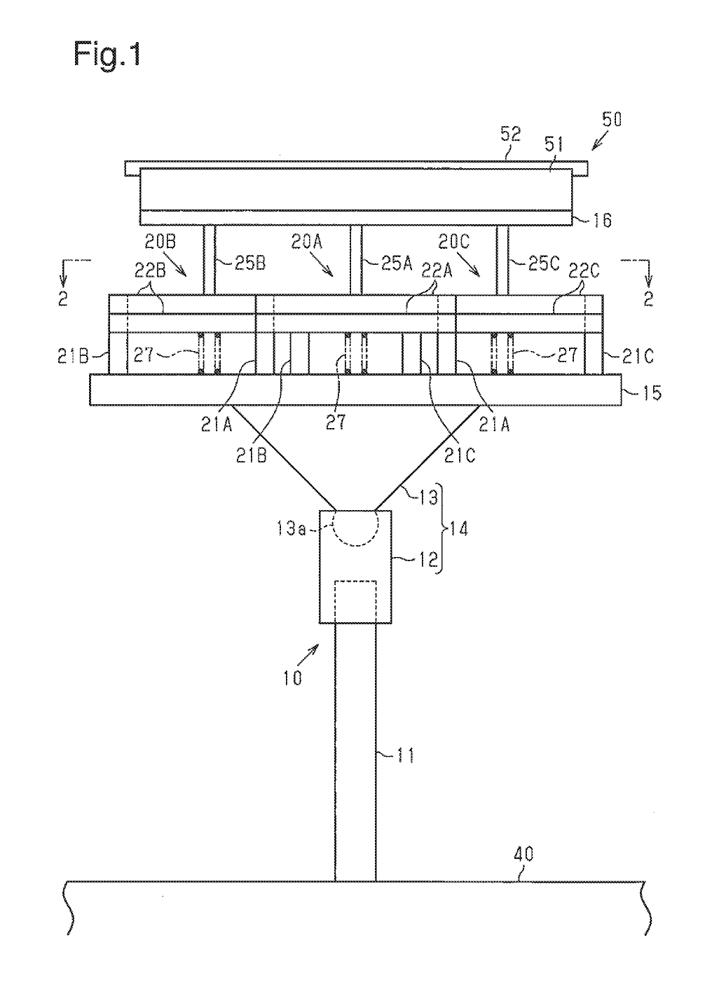

An operation training apparatus according to a first embodiment will now be described with reference to FIGS. 1 to 7.

The operation training apparatus of the first embodiment is used in training for cardiac blood vessel anastomosis, or, more specifically, beating-heart coronary artery bypass surgery. The operation training apparatus is arranged in a case (not shown) having an opening in its upper section.

As shown in FIG. 1, a support mechanism 10 is fixed to a bottom wall 40 of the case.

The support mechanism 10 includes a tubular leg member 11, a tubular lower member 12, an upper member 13, and a support plate 15. The leg member 11 extends upward from the bottom wall 40. The lower member 12 is arranged on the leg member 11. The upper member 13 is arranged on the lower member 12. The support plate 15 substantially has a square shape as viewed from above and is fixed to an upper section of the upper member 13.

The leg member 11 has an upper section that is inserted in the lower member 12 through a lower opening of the lower member 12. This allows the lower member 12 to ascend and descend with respect to the leg member 11. The lower member 12 has a screw (not shown) for fixing the lower member 12 to the leg member 11.

A spherical portion 13a is formed integrally with the lower end of the upper member 13. The spherical portion 13a is pivotally supported in the interior of the lower member 12. This allows the upper member 13 to incline about the spherical portion 13a with respect to the lower member 12. That is, the lower member 12 and the upper member 13 configure a universal joint 14. The lower member 12 has a screw (not shown) for fixing the upper member 13 to the lower member 12.

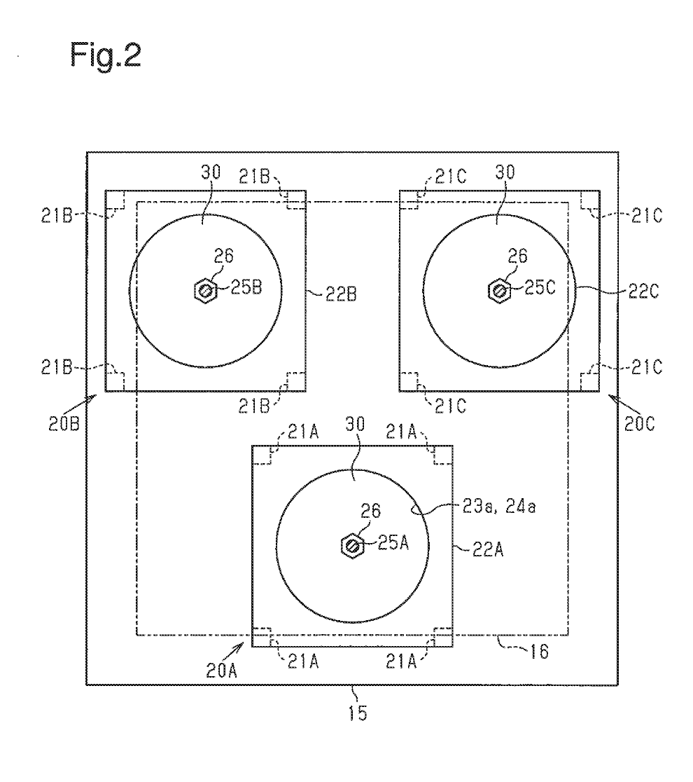

A model body 50 mimics a section of the heart. As shown in FIGS. 1 and 2, three driving devices 20A, 20B, 20C are fixed onto the support plate 15 to move the model body 50 in a manner mimicking expansion-contraction of the heart, that is, pulsation.

The driving devices 20A, 20B, and 20C include bolts 25A, 25B, and 25C, respectively, each as an upwardly extending output member.

A holding plate 16 is fixed to the upper ends of the bolts 25A, 25B, 25C and substantially has a rectangular shape as viewed from above. The model body 50 is fixed to the upper surface of the holding plate 16.

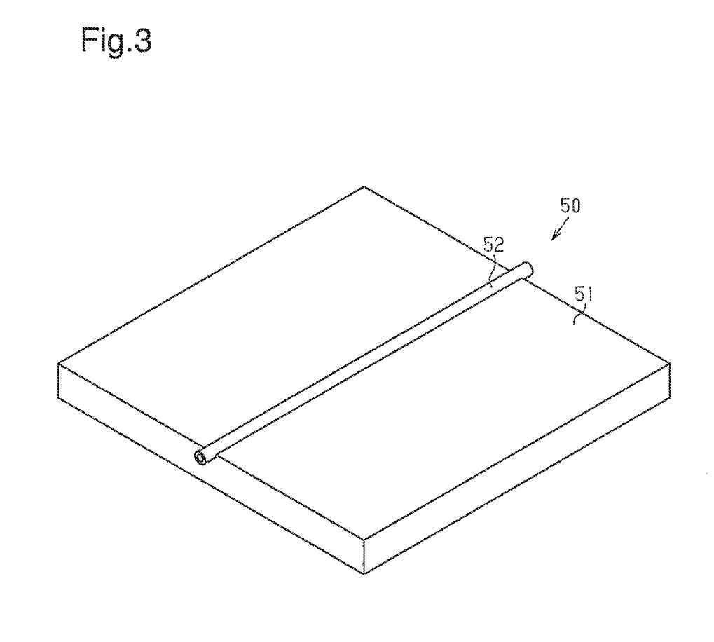

With reference to FIGS. 1 and 3, the model body 50 mimics a section of living tissue on which training is carried out, or, specifically, a section of the heart surface on which the coronary artery is exposed.

As shown in FIG. 3, the model body 50 has a myocardium model 51 and a blood vessel model 52. The myocardium model 51 is shaped substantially like a rectangular parallelepiped. The blood vessel model 52 is fixed to a middle section of the upper surface of the myocardium model 51 in the width direction. The blood vessel model 52 extends in the longitudinal direction of the myocardium model 51. The myocardium model 51 and the blood vessel model 52 are each formed by an elastic member made of, for example, silicone elastomer.

The training for coronary artery bypass surgery according to the first embodiment involves sectioning a certain part of the blood vessel model 52 and inosculating an end of another blood vessel model (not shown) to the sectioned part.

The configuration of each of the driving devices 20A, 20B, 20C in the first embodiment will now be described in detail.

Specifically, the driving devices 20A, 20B, 20C are configured to be identical. Therefore, the description below is focused on the configuration of the driving device 20A to omit repeated description. Some components of the driving devices 20B, 20C are given the same reference numerals as those of the corresponding components of the driving device 20A. Other components are given numerals obtained by replacing the letter A in 2*A with letters B or C.

As illustrated in FIGS. 1, 2, and 4, the driving device 20A includes two frame members 22A and four support members 21A. The frame members 22A each substantially have a square shape as viewed from above and are fixed to each other in a stacked state in the up-down direction. The upper ends of the support members 21 are fixed to the four corner portions of the lower one of the frame members 22A. The lower ends of the support members 21 are fixed to the upper surface of the support plate 15. That is, the support members 21 support the frame members 22A.

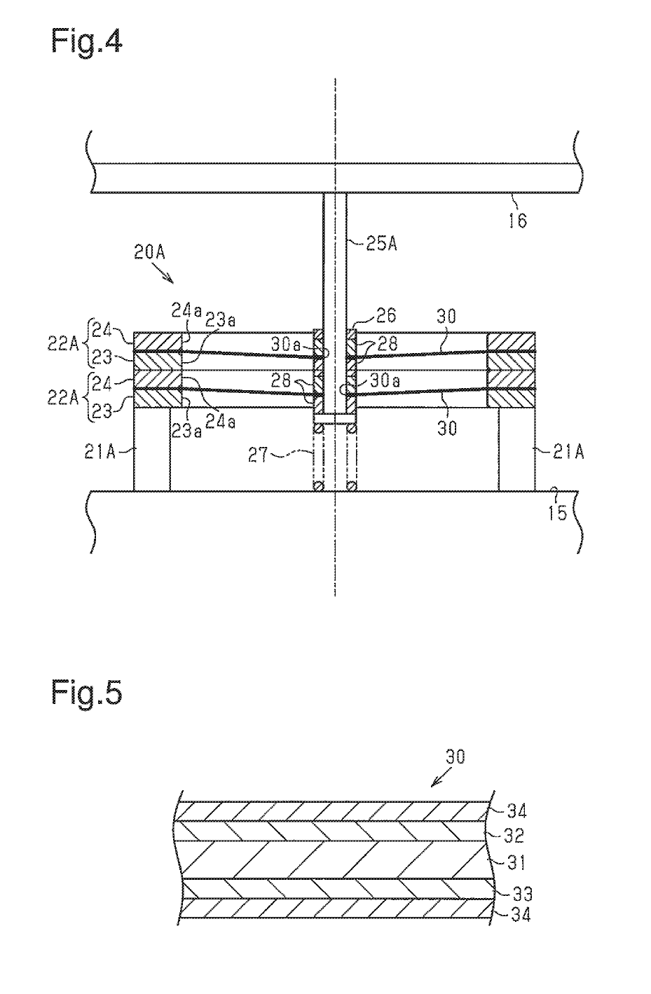

With reference to FIG. 4, each frame member 22A is formed by a pair of frame portions (a lower frame portion 23 and an upper frame portion 24). The frame portions 23, 24 are fixed to each other in a stacked state in the up-down direction. The lower frame portion 23 and the upper frame portion 24 are identical with each other and have a central hole 23a and a central hole 24a, respectively. The central holes 23a, 24a each have a circular shape as viewed from above. The lower and upper frame portions 23, 24 are both made of hard plastic.

A sheet-like dielectric actuator 30 is sandwiched between the lower frame portion 23 and the upper frame portion 24 and substantially has a square shape as viewed from above.

As shown in FIG. 5, the dielectric actuator 30 (dielectric elastomer actuator: DEA) is a piezoelectric element made of elastomer that has a sheet-like dielectric layer 31, a pair of electrode layers 32, 33, and insulating layers 34. The dielectric layer 31 is made of dielectric elastomer. The electrode layers 32, 33 are made of conductive elastomer and sandwich the dielectric layer 31 from the opposite sides in the thickness direction of the dielectric layer 31. The insulating layers 34 sandwich the electrode layers 32, 33 from the opposite sides in the thickness direction of the electrode layers 32, 33.

In the first embodiment, the dielectric layer 31 is made of dielectric elastomer containing cross-linked polyrotaxane. Specifically, the dielectric elastomer consists of polyethylene glycol as the straight-chain molecule, cyclodextrin as the cyclic molecule, and adamantanamine as the blocking group.

Further, in the first embodiment, each of the electrode layers 32, 33 is made of conductive elastomer containing insulating polymer and conductive filler. Polyrotaxane is used as the insulating polymer and Ketjen black (registered trademark) is used as the conductive filler.

The thickness of the dielectric layer 31 and the thickness of each electrode layer 32, 33 are both several tens to hundreds of micrometers.

With reference to FIG. 4, a through-hole 30a is formed at the center of each dielectric actuator 30. The common bolt 25A is inserted through the through-holes 30a from below. A nut 26 is threaded onto a section of the bolt 25A projecting upward from the through-hole 30a of the upper one of the dielectric actuators 30. Spacers 28 are arranged between the head of the bolt 25A and the lower dielectric actuator 30, between the upper and lower dielectric actuators 30, and between the upper dielectric actuator 30 and the nut 26. In this manner, the bolt 25A is fixed to the dielectric actuators 30.

An urging member 27 is arranged between the basal end (the lower end) of the bolt 25A and the support plate 15 and urges the bolt 25A (the dielectric actuators 30) downward. In the first embodiment, a coil spring is used as the urging member 27.

Specifically, referring to FIG. 4, each dielectric actuator 30 is held in a flat state when receiving no voltage and being maintained in equilibrium with the downward urging force of the urging member 27.

As shown in FIG. 2, the bolts 25A, 25B, 25C of the driving devices 20A, 20B, 20C apply force to different sections of the model body 50.

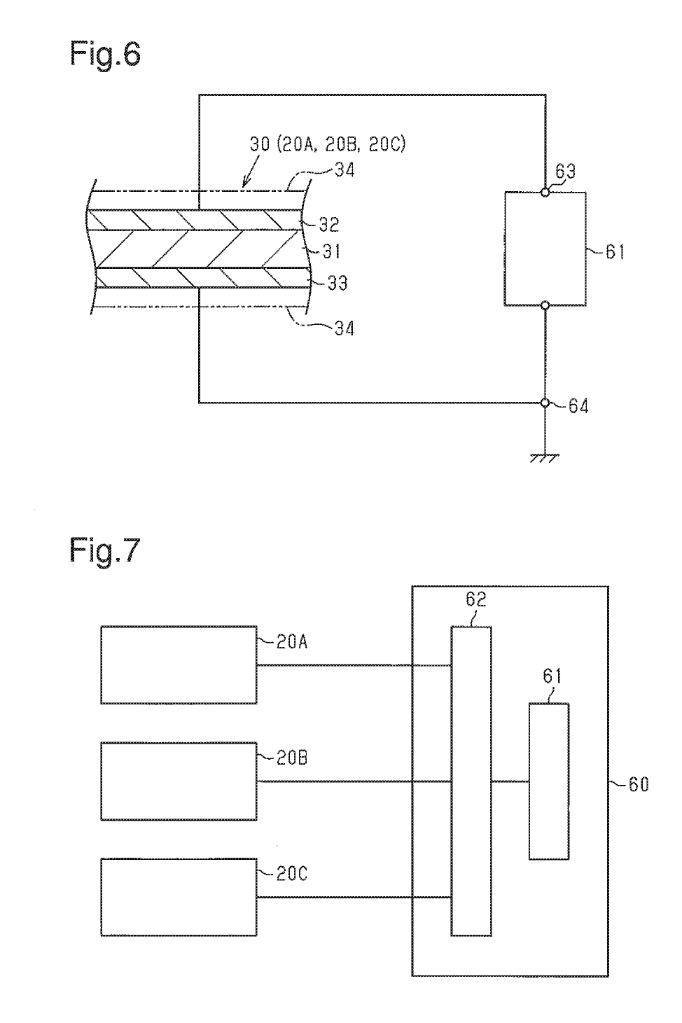

With reference to FIGS. 6 and 7, a controller 60 is electrically connected to the driving devices 20A, 20B, 20C and controls the manners in which the driving devices 20A, 20B, 20C are driven. That is, referring to FIG. 6, a positive terminal 63 of a power source 61 is connected to the positive electrode layer 32 of each dielectric actuator 30 through a lead. Also, a grounding terminal 64 of the power source 61 is connected to the negative electrode layer 33 of each dielectric actuator 30 through another lead.

As shown in FIG. 7, the controller 60 includes a control section 62. The control section 62 controls the manner in which the power source 61 applies a DC voltage of, for example, 900 to 1500 V between the electrode layers 32, 33 of each dielectric actuator 30. The control section 62 variably sets the voltage to be applied in the frequency range of 0 to 5 Hz, thus driving the dielectric actuators 30 in the range of 0 to 300 beats per minute (BPM). Next, the operation of the first embodiment will be described.

Prior to starting the training for coronary artery bypass surgery using the operation training apparatus of the first embodiment, the support mechanism 10 is manipulated to adjust the height and angle of the holding plate 16 (the model body 50).

During the training, the controller 60 controls the manner in which electric power is supplied to each of the driving devices 20A, 20B, 20C. In this manner, the driving manner of each driving device 20A, 20B, 20C is controlled to control the movement of the model body 50.

That is, when the DC voltage is applied between the positive electrode layer 32 and the negative electrode layer 33 of each dielectric actuator 30 of each driving device 20A, 20B, 20C, the force that moves the positive charge and the negative charge toward each other is produced in the interior of the dielectric actuator 30. The force compresses the dielectric layer 31 in the thickness direction, thus expanding the dielectric layer 31 in a direction along the surface of the dielectric layer 31. At this time, the thickness of the dielectric actuator 30 is decreased so that the elastic force of the dielectric actuator 30 becomes smaller than the urging force of the urging member 27. As a result, the dielectric actuator 30 is displaced downward.

Thereafter, when the voltage applied to the dielectric actuator 30 is stopped, the original thickness of the dielectric layer 31 is restored, thus restoring the elastic force of the dielectric actuator 30. This displaces the dielectric actuator 30 upward.

Particularly, in the first embodiment, voltage is applied to the dielectric actuators 30 of the driving devices 20A, 20B, 20C in different phases.

As a result, the dielectric actuators 30 of the driving device 20A, the dielectric actuators 30 of the driving device 20B, and the dielectric actuators 30 of the driving device 20C are displaced in the up-down direction in different phases. The bolts 25A, 25B, 25C, which are arranged in the central sections of the corresponding dielectric actuators 30, are thus moved in the up-down direction in different phases. In this manner, the model body 50, which is held by the holding plate 16, is moved in a manner mimicking the expansion-contraction of the heart.

In the first embodiment, each of the driving devices 20A, 20B, 20C includes the dielectric actuators 30. This increases the movement speed, that is, improves the response, of the model body 50, compared to a case with a conventional actuator made of shape memory alloy. The operation training apparatus thus can be adapted for training for operations on models of high-heart-rate patients, such as children.

The operation training apparatus of the above-described first embodiment has the following advantages.

(1) The operation training apparatus is used in training for beating-heart coronary artery bypass surgery and includes the model body 50, which mimics a section of the heart, the driving devices 20A, 20B, 20C, and the controller 60. The driving devices 20A, 20B, 20C move the model body 50 in a manner mimicking the expansion-contraction of the heart. The controller 60 controls the driving manner of each of the driving devices 20A, 20B, 20C. Each driving device 20A, 20B, 20C includes the dielectric actuators 30. Each of the dielectric actuators 30 has the dielectric layer 31 and the two electrode layers 32, 33. The dielectric layer 31 is made of dielectric elastomer. The electrode layers 32, 33 are made of conductive elastomer and sandwich the dielectric layer 31.

This configuration operates in the above-described manners and thus can be adapted for training for operations on models of high-heart-rate patients, such as children. The operation training apparatus is thus adapted for training for a wide variety of operations.

(2) The dielectric layer 31, which is a component of each dielectric actuator 30, contains cross-linked polyrotaxane.

In this configuration, each dielectric actuator 30 is made of slide-ring material. This substantially cancels hysteresis loss at the time of deformation of the dielectric actuator 30. As a result, the movement re-productivity by the model body 50 is improved. Also, since the slide-ring material has freely movable cross-link points, the durability of each dielectric actuator 30 is enhanced. This improves the durability of each driving device 20A, 20B, 20C.

(3) The operation training apparatus includes the three driving devices 20A, 20B, 20C. The driving devices 20A, 20B, 20C apply force to different sections of the model body 50.

In this configuration, by operating the driving devices 20A, 20B, 20C in different manners by means of the controller 60, the model body 50 can be moved in complicated manners. This allows the model body 50 to move in a manner imitating the actual expansion-contraction of the heart.

(4) Each dielectric actuator 30 is shaped like a sheet having a surface. The dielectric actuator 30 has the associated bolt 25A, 25B, 25C (the associated output member). The bolt 25A, 25B, 25C extends in a direction perpendicular to the surface of the dielectric actuator 30 and applies force to the holding plate 16 (the model body 50).

In this configuration, by setting the positions at which the bolts 25A, 25B, 25C (the output members) apply force to the model body 50 as needed, the manner in which the model body 50 is moved is easily changed.

### Second Embodiment

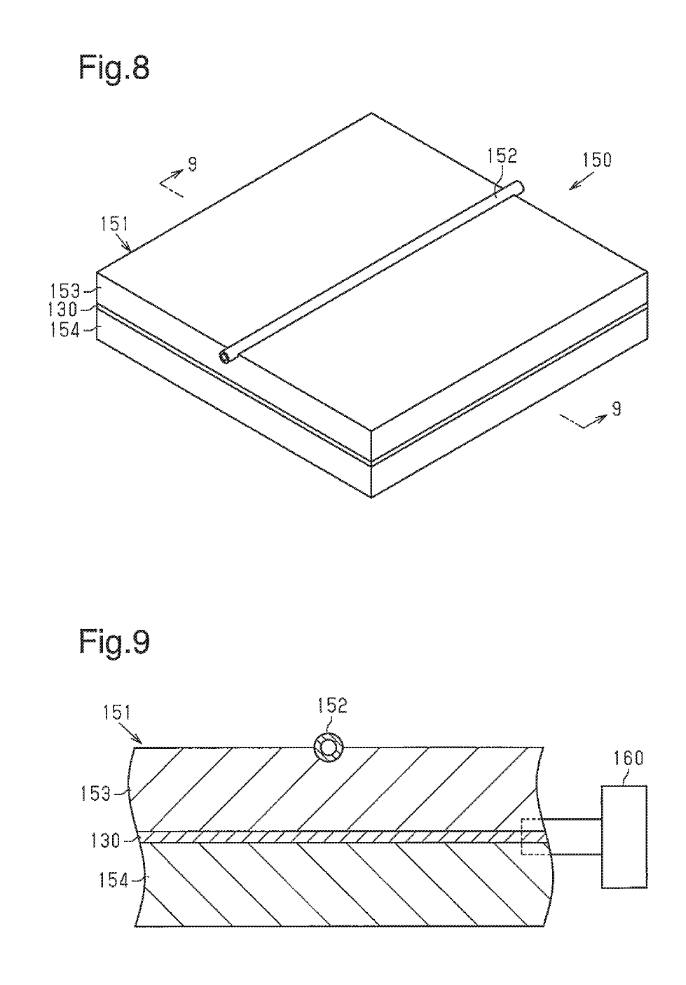

An operation training apparatus according to a second embodiment will hereafter be described with reference to FIGS. 8 and 9.

As shown in FIGS. 8 and 9, a model body 150 of the second embodiment mimics a section of the heart surface on which the coronary artery is exposed, like the model body 50 of the first embodiment. The description below is focused on the difference between the model body 150 of the second embodiment and the model body 50 of the first embodiment. Reference numerals that are obtained by adding “100” to reference numerals of components of the first embodiment are given to the components that correspond to the components of the first embodiment and redundant description is omitted.

The model body 150 has a myocardium model 151 and a blood vessel model 152. The myocardium model 151 has an upper base 153 and a lower base 154. Each of the upper and lower bases 153, 154 is shaped substantially like a rectangular parallelepiped. The blood vessel model 152 is fixed to the upper surface of the upper base 153. The myocardium model 151, the upper base 153, and the lower base 154 are each formed by an elastic member made of, for example, silicone elastomer.

A sheet-like dielectric actuator 130 is fixed between the upper base 153 and the lower base 154 using adhesive. The dielectric actuator 130 is identical with the dielectric actuator 30 of the first embodiment.

A controller 160 is electrically connected to the dielectric actuator 130 and controls the manner in which the dielectric actuator 130 is driven.

The operation of the operation training apparatus of the second embodiment will hereafter be described.

The controller 160 controls the manner in which electric power is supplied to the dielectric actuator 130, thus controlling the driving manner of the dielectric actuator 130 and the movement of the blood vessel model 152.

That is, the upper base 153 is expanded and contracted by means of the sheet-like dielectric actuator 130. Also, the blood vessel model 152, which is fixed to the upper base 153, is expanded and contracted. As a result, the blood vessel model 152 is pulsated in correspondence with the heart rate.

The operation training apparatus of the above-described second embodiment achieves the following advantages in addition to the advantage (2) of the first embodiment.

(5) The operation training apparatus is used in training for operations on pulsating human blood vessels and includes the blood vessel model 152, the dielectric actuator 130, and the controller 160. The blood vessel model 152 mimics the blood vessel. The dielectric actuator 130 serves as a driving device that moves the blood vessel model 152 in a manner mimicking the pulsation. The controller 160 controls the driving manner of the dielectric actuator 130.

This configuration can be adapted for training for operations on a pulsating blood vessel as in coronary artery bypass surgery. Particularly, since the driving device is the dielectric actuator 130, the movement speed of the blood vessel model 152 is increased, that is, the response of the blood vessel model 152 is improved. The operation training apparatus is thus adapted for training for operations on models of high-heart-rate patients such as children. As a result, the operation training apparatus is adapted for training for a wider variety of operations.

(6) The operation training apparatus includes the upper base 153 to which the blood vessel model 152 is fixed. The dielectric actuator 130 is shaped like a sheet and fixed to the upper base 153.

In this configuration, while the upper base 153 is expanded and contracted by means of the sheet-like dielectric actuator 130, the blood vessel model 152, which is fixed to the upper base 153, is also expanded and contracted. This facilitates the movement of the blood vessel model 152 in the pulsation mimicking manner.

### Third Embodiment

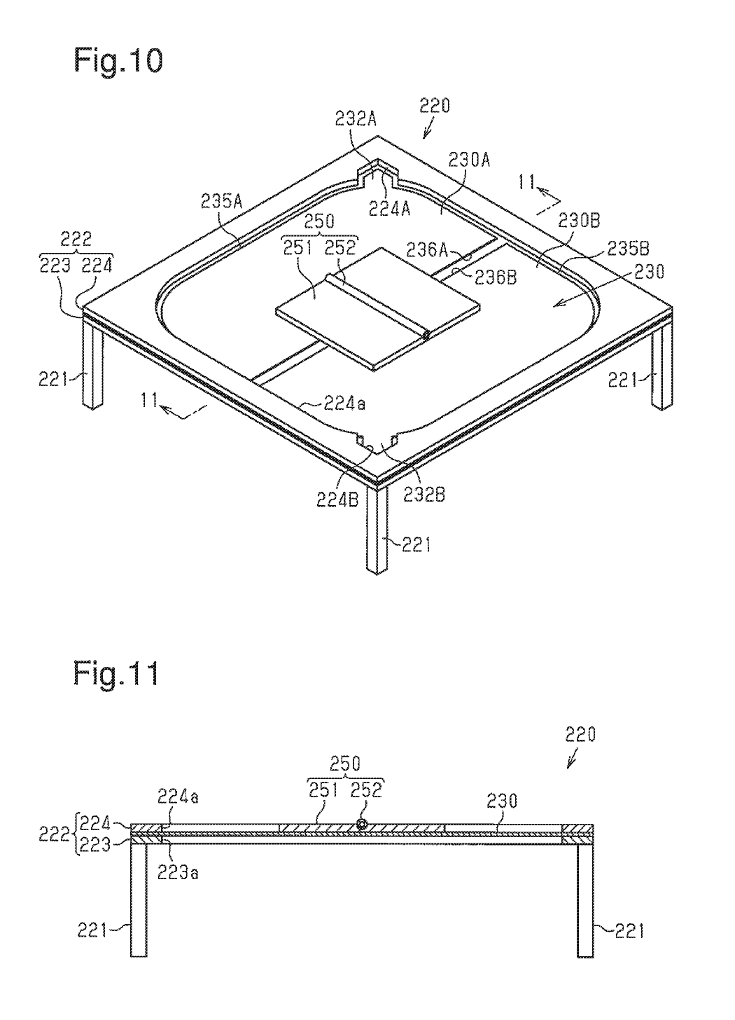

An operation training apparatus of a third embodiment will now be described with reference to FIGS. 10 to 14, mainly on the difference between the first embodiment and the third embodiment. In the first embodiment, mainly the urging force of the urging member 27 (the coil spring) is used as the driving force that displaces the model body 50 downward. In contrast, in the third embodiment, only the gravitational force acting on the model body is used as the aforementioned driving force. Reference numerals that are obtained by adding “200” to reference numerals of components of the first embodiment are given to the components that correspond to the components of the first embodiment and redundant description is omitted.

As shown in FIGS. 10 and 11, a driving device 220 includes a frame member 222 formed by a lower frame portion 223 and an upper frame portion 224. The lower and upper frame portions 223, 224 each have a square plate-like shape as viewed from above. The lower and upper frame portions 223, 224 are fixed to each other in a stacked state in the up-down direction.

The frame portions 223 and 224 have central holes 223a and 224a, respectively. Each of the central holes 223a, 224a has a square shape with round corners as viewed from above.

Four support members 221 each extend downward from one of the four corner sections of the lower frame portion 223.

With reference to FIG. 10, a pair of recesses 224A, 224B is formed in a diagonally opposed pair of the corner sections of the square shape defined by the upper frame portion 224 on the inner peripheral edge of the central hole 224a of the upper frame portion 224.

Also, a pair of recesses (not shown) is formed in a diagonally opposed pair of the corner sections of the square shape defined by the lower frame portion 223 on the inner peripheral edge of the central hole 223a of the lower frame portion 223. These corner sections do not correspond to the diagonally opposed corner sections in which the recesses 224A, 224B of the upper frame portion 224 are located.

As illustrated in FIGS. 10 and 11, a peripheral edge section of a soft sheet-like dielectric actuator 230 (a soft membrane) is fixed between the lower frame portion 223 and the upper frame portion 224 using adhesive. The dielectric actuator 230 substantially has a square shape, as viewed from above.

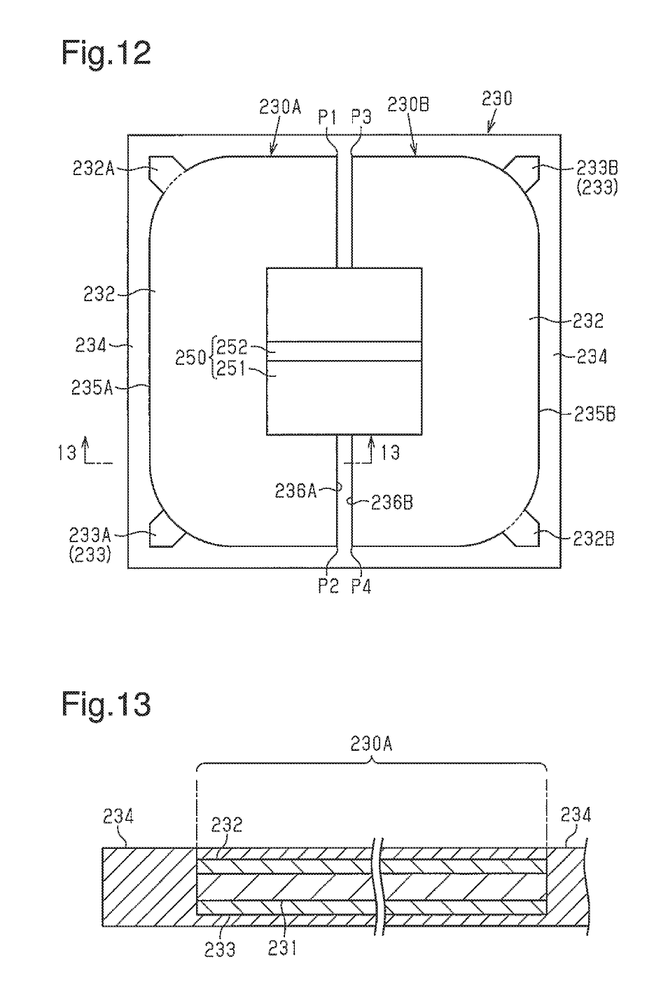

Referring to FIGS. 12 and 13, the dielectric actuator 230 is a piezoelectric element made of elastomer like the dielectric actuator 30 of the first embodiment. The dielectric actuator 230 includes two driving layers 230A, 230B and a common insulating layer 234 for the driving layers 230A, 230B. The driving layers 230A, 230B are arranged on the same plane. The insulating layer 234 sandwiches the driving layers 230A, 230B from opposite sides in the thickness direction of each of the driving layers 230A, 230B. Each driving layer 230A, 230B includes a dielectric layer 231 and a pair of electrode layers (a positive electrode layer 232 and a negative electrode layer 233). The positive and negative electrode layers 232, 233 sandwich the dielectric layer 231. The insulating layer 234 is transparent. Therefore, in FIGS. 10 and 12, the driving layers 230A, 230B, which are located in the interior of the insulating layer 234, are represented by solid lines.

As shown in FIGS. 10 and 12, the driving layer 230A (230B) has a substantially U-shaped outer peripheral edge section 235A (235B) and an opposed edge section 236A (236B). The outer peripheral edge section 235A (235B) is located peripherally inward from and extends along the inner peripheral edge of the central hole 223a. The opposed edge section 236A (236B) extends linearly between opposite ends P1, P2 (P3, P4) of the outer peripheral edge section 235A (235B). The opposed edge section 236A of the driving layer 230A and the opposed edge section 236B of the driving layer 230B are spaced apart and opposed to each other.

With reference to FIGS. 12 and 13, the outer peripheral edge section of the dielectric actuator 230 and the zone between the driving layers 230A, 230B are configured simply by the insulating layer 234.

As illustrated in FIGS. 10 and 12, a projected piece 232A is formed in the section of the outer peripheral edge section 235A of the driving layer 230A that corresponds to the recess 224A of the upper frame portion 224. The projected piece 232A is configured by the section of the positive electrode layer 232 that projects peripherally outward. Also, a projected piece 233A is formed in the section of the outer peripheral edge section 235A of the driving layer 230A that corresponds to the recess (not shown) of the lower frame portion 223. The projected piece 233A is configured by the section of the negative electrode layer 233 that projects peripherally outward.

A projected piece 232B is formed in the section of the outer peripheral edge section 235B of the driving layer 230B that corresponds to the recess 224B of the upper frame portion 224. The projected piece 232B is configured by the section of the positive electrode layer 232 that projects peripherally outward. Also, a projected piece 233B is formed in the section of the outer peripheral edge section 235B of the driving layer 230B that corresponds to the recess (not shown) of the lower frame portion 223. The projected piece 233B is configured by the section of the negative electrode layer 233 that projects peripherally outward.

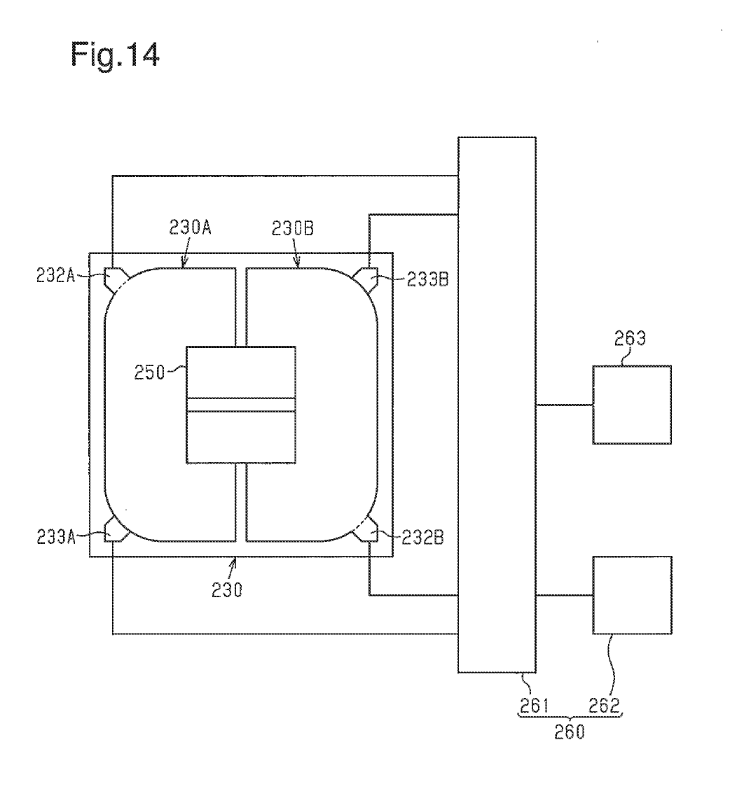

As shown in FIG. 14, each of the projected pieces 232A, 233A (232B, 233B) has a contact with respect to a control substrate 261. The control substrate 261 controls the manner in which the dielectric actuator 230 is driven.

Referring to FIGS. 10 to 12, a model body 250 is fixed to a central section of the upper surface of the dielectric actuator 230.

Like the model body 50 of the first embodiment, the model body 250 mimics a section of living tissue on which training is carried out, or, specifically, a section of the heart surface on which the coronary artery is exposed.

The model body 250 has a myocardium model 251 and a cylindrical blood vessel model 252. The myocardium model 251 is formed like a soft sheet that has a square shape having an outer peripheral length smaller than the inner peripheral length of the central hole 223a, as viewed from above. The blood vessel model 252 is fixed to a middle section of the upper surface of the myocardium model 251 in the width direction and extends in the direction defined by the length of the myocardium model 251. The myocardium model 251 and the blood vessel model 252 are both formed by an elastic member made of, for example, silicone elastomer.

The myocardium model 251 is supported in a state in which the whole myocardium model 251 is stacked on the two adjacent driving layers 230A, 230B in a manner spreading across the driving layers 230A, 230B. In the third embodiment, the myocardium model 251 is fixed onto the insulating layer 234 using adhesive. The model body 250 is supported substantially only through the tension of the dielectric actuator 230.

The thickness of the model body 250 is, for example, 2 mm.

Specifically, the model body 250 is arranged in an area that is 20 mm in diameter in the dielectric actuator 230. It is preferable that the dielectric actuator 230 have such an elastic property that, when a load of 10 g is applied to the area, the area is displaced downward by an amount of 1 to 100 mm. The dielectric actuator 230 of the third embodiment has such an elastic property that the aforementioned area is displaced downward by an amount of approximately 10 mm.

It is also preferable that Young's modulus of the dielectric actuator 230 be 1 to 3 MPa.

The training for coronary artery bypass surgery according to the third embodiment involves sectioning a certain part of the blood vessel model 252 and inosculating an end of another blood vessel model (not shown) to the sectioned part.

As illustrated in FIG. 14, a controller 260 is electrically connected to the driving layers 230A, 230B of the dielectric actuator 230 and controls the manners in which the driving layers 230A, 230B are driven. The controller 260 includes the control substrate 261 and a terminal device 262, such as a tablet terminal. A power source 263 is also electrically connected to the control substrate 261.

The control substrate 261 controls the manner in which the power source 263 applies a DC voltage of, for example, 900 to 1500 V between the electrode layers 232, 233 of each driving layer 230A, 230B in correspondence with an input signal from the terminal device 262. Specifically, the control substrate 261 variably sets the voltage to be applied in the frequency range of 0 to 5 Hz, thus driving the driving layers 230A, 230B in the range of 0 to 300 BPM.

The basic operation of the operation training apparatus of the third embodiment will hereafter be described.

During the training for coronary artery bypass surgery using the operation training apparatus of the third embodiment, the controller 260 controls the manner in which electric power is supplied to the driving layers 230A, 230B. In this manner, the driving manner of each driving layer 230A, 230B is controlled to control the movement of the model body 250.

That is, when the DC voltage is applied between the positive electrode layer 232 and the negative electrode layer 233 of the driving layer 230A (230B), the force that moves the positive charge and the negative charge toward each other is produced in the interior of the driving layer 230A (230B). The force compresses the dielectric layer 231 in the thickness direction, thus expanding the dielectric layer 231 in a direction along the surface of the dielectric layer 231. This decreases the elastic force of the driving layer 230A (230B) such that the central section of the driving layer 230A (230B) is displaced downward due to the own weight.

Thereafter, when the voltage applied to the driving layer 230A (230B) is stopped, the original thickness of the dielectric layer 231 is restored, thus restoring the elastic force of the driving layer 230A (230B). This displaces the driving layer 230A (230B) upward.

Particularly, in the third embodiment, the voltage is applied to the driving layers 230A, 230B in different phases.

In this manner, the driving layers 230A, 230B ascend and descend in an alternating manner. This moves the model body 250, which is arranged to spread across the driving layers 230A, 230B, in a manner mimicking the expansion-contraction of the heart.

The operation training apparatus of the above-described third embodiment achieves the following advantages in addition to the advantages (1) and (2) of the first embodiment.

(7) The dielectric actuator 230 and the model body 250 are both formed like a soft sheet. The model body 250 is supported in a state in which the whole model body 250 is stacked on the dielectric actuator 230.

In a case of a thick and hard model body, the movement of the whole model body is limited, for example, if a section of the model body is pressed by forceps while the model body is moving. Therefore, the model body may be incapable of moving in a manner imitating the actual expansion-contraction of the heart.

However, in the above-described configuration, regardless of which section of the model body 250 is pressed by forceps while the model body 250 is moving, non-pressed sections of the model body 250 continuously move together with the dielectric actuator 230, which is formed like a soft sheet. Meanwhile, the force by which the forceps press the model body 250 causes the dielectric actuator 230 to expand, thus moving the section of the model body 250 to which the force is applied. This allows the model body 250 to move in a manner imitating the actual expansion-contraction of the heart.

Further, regardless of which section of the model body 250 is pressed by forceps, for example, the model body 250 and the dielectric actuator 230 smoothly move about the pressed section in the pressing direction. This allows the model body 250 to move in a manner imitating the actual movement of the heart more accurately.

(8) The dielectric actuator 230 includes the two driving layers 230A, 230B, which are arranged on the same plane, and the common insulating layer 234 for the driving layers 230A, 230B. Each of the driving layers 230A, 230B has the dielectric layer 231 and a pair of electrode layers 232, 233, which sandwiches the dielectric layer 231. The insulating layer 234 sandwiches the driving layers 230A, 230B from the opposite sides in the thickness direction of the driving layers 230A, 230B. The model body 250 is arranged on the insulating layer 234 in a manner spreading across the driving layers 230A, 230B.

This configuration allows the two driving layers 230A, 230B to apply force to different sections of the model body 250. Therefore, by operating the driving layers 230A, 230B in different manners by means of the controller, the model body 250 is moved in complicated manners. This allows the model body 250 to move in a manner imitating the actual expansion-contraction of the heart more accurately.

The operation training apparatus of the third embodiment has the following advantages.

(A) The operation training apparatus is an apparatus used in training for operations on the heart and includes the model body 250, which mimics a section of the heart, and the dielectric actuator 230 as a soft membrane, which supports the model body 250.

In this configuration, the model body 250 is supported by the dielectric actuator 230, which is a soft membrane. Therefore, if the model body 250 is pressed by forceps, for example, the dielectric actuator 230 expands, thus moving the model body 250 smoothly in the pressing direction. This allows the model body 250 to move in a manner imitating the actual movement of the heart.

(B) The model body 250 is formed like a soft sheet and supported in a state stacked on the dielectric actuator 230.

In this configuration, the model body 250 is also formed like a soft sheet. Therefore, if a section of the model body 250 is pressed by forceps, for example, the model body 250 and the dielectric actuator 230 smoothly move about the pressed section in the pressing direction. This allows the model body 250 to move in a manner imitating the actual movement of the heart more accurately.

(C) The dielectric actuator 230 has such an elastic property that, when a load of 10 g is applied to the area that is 20 mm in diameter in which the model body 250 is arranged in the dielectric actuator 230, the area is displaced downward by an amount of 1 to 100 mm.

For example, if a load of 10 g is applied to the aforementioned area in the dielectric actuator 230 and the area is displaced downward by an amount smaller than 1 mm, it is indicated that the dielectric actuator 230 is excessively hard. In this case, the movement of the model body 250 tends to become unnatural.

In contrast, if the aforementioned area in the dielectric actuator 230 is displaced downward by an amount greater than 100 mm, it is indicated that the dielectric actuator 230 is excessively soft. In this case, the movement of the model body 250 tends to become unnatural.

However, the above-described configuration allows the dielectric actuator 230 to have an elastic property that is substantially similar to the actual elastic property of the heart.

(D) The operation training apparatus includes the driving device 220. The driving device 220 moves the model body 250 in a manner mimicking the expansion-contraction of the heart. The driving device 220 includes the dielectric actuator 230 as a soft membrane.

In this configuration, the dielectric actuator 230, which is a component of the driving device 220, is operated in a manner mimicking the expansion-contraction of the heart. This allows the model body 250 to move in a manner imitating the actual expansion-contraction of the heart.

(E) The driving device 220 includes the soft sheet-like dielectric actuator 230. The dielectric actuator 230 includes the dielectric layer 231 and a pair of electrode layers 232, 233. The dielectric layer 231 is made of dielectric elastomer. The electrode layers 232, 233 are made of conductive elastomer and sandwich the dielectric layer 231.

In the above-described configuration, the driving device 220 includes the soft sheet-like dielectric actuator 230. This increases the movement speed of the model body 250, that is, improves the response of the model body 250, compared to the aforementioned conventional actuator made of shape memory alloy. The operation training apparatus thus can be adapted for training for operations on models of high-heart-rate patients such as children. As a result, the operation training apparatus is adapted for training for a wide variety of operations. Also, through such a simple configuration, the driving device 220 that moves the model body 250 in a stable manner is embodied.

<Modifications>

The above-described embodiments may be modified as follows. The above-described embodiments and the following modifications can be combined as long as the combined modifications remain technically consistent with each other.

In the first embodiment, the operation training apparatus includes the three driving devices 20A, 20B, 20C. However, the operation training apparatus may employ four or more driving devices, two driving devices, or a single driving device.

The urging member 27 may be omitted.

In the first embodiment, the two dielectric actuators 30 are arranged in each of the driving devices 20A, 20B, 20C and spaced apart in the up-down direction. However, three or more dielectric actuators 30 may be arranged in each driving device 20A, 20B, 20C and spaced apart in the up-down direction. Alternatively, multiple (for example, fifty) dielectric actuators 30 may be tightly stacked together to configure a driving device. In this case, the urging member 27 may be omitted. Further alternatively, a single dielectric actuator 30 may configure a driving device.

In the third embodiment, the dielectric actuator 230 has the two driving layers 230A, 230B. However, a dielectric actuator including three or more driving layers or a dielectric actuator including a single driving layer may be employed.

The dielectric elastomer forming each dielectric layer 31, 231 is not restricted to polyrotaxane but may be another type of dielectric elastomer, such as silicone elastomer, acrylic elastomer, or urethane elastomer.

The insulating polymer in the conductive elastomer forming each electrode layer 32, 33, 232, 233 is not restricted to polyrotaxane but may be another type of insulating polymer, such as silicone elastomer, acrylic elastomer, or urethane elastomer. Also, one of the listed insulating polymers may be used independently or multiple types of these insulating polymers may be used in combination.

The conductive filler in the conductive elastomer forming each electrode layer 32, 33, 232, 233 is not restricted to Ketjen black but may be another type of carbon black or metal particles, such as copper particles or silver particles. Also, one of the listed conductive fillers may be used independently or multiple types of these conductive fillers may be used in combination.

The driving device is not restricted to a driving device including a sheet-like dielectric actuator but may be a tubular driving device having a sheet-like dielectric actuator rolled in a tubular shape. In this case, the driving device expands and contracts in the axial direction of the tubular dielectric actuator.

The driving device is not restricted to a driving device including a dielectric actuator but may be, for example, other electroactive polymer actuators (EPAs) including ionic polymer metal composite (IPMC). Also, in the illustrated embodiments, the electroactive polymer actuator is configured by a dielectric actuator having a dielectric layer that is made of dielectric elastomer and a pair of electrode layers that is made of conductive elastomer and sandwiches the dielectric layer. As a result, through such a simple configuration, a driving device that moves a model body in a stable manner is embodied.

The soft membrane (the dielectric actuator 230) in the third embodiment is not restricted to an electroactive polymer actuator. The soft membrane may be, for example, an elastomer membrane having the elastic property of the third embodiment. Also, other actuators such as an actuator having wires made of shape memory alloy may be employed to reciprocate a frame member that supports the soft membrane in the up-down direction.

The operation training apparatus of the third embodiment may be employed in training for operations on living tissue that does not expand or contract.

The operation training apparatuses according to the present disclosure are not restricted to the use in training for beating-heart coronary artery bypass surgery but may be used in training for operations on expanding and contracting living tissue other than that of the heart, as in catheterization.

The present examples and embodiments are to be considered as illustrative and not restrictive, and the disclosure is not to be limited to the details given herein, but may be modified within the scope and equivalence of the appended claims.

1. An operation training apparatus used in training for operation on living tissue that expands and contracts, the apparatus comprising:
a model body, which mimics the living tissue;
a driving device, which moves the model body in a manner mimicking the expansion-contraction; and
a controller, which controls a manner in which the driving device is driven,
wherein the driving device includes an electroactive polymer actuator.

2. The operation training apparatus according to claim 1, wherein
the driving device is one of a plurality of driving devices, and
the driving devices apply force to different sections of the model body.

3. The operation training apparatus according to claim 1, wherein
the electroactive polymer actuator is shaped like a sheet having a surface, and
the electroactive polymer actuator has an output member, which extends in a direction perpendicular to the surface and applies force to the model body.

4. An operation training apparatus used in training for operations on a pulsating blood vessel, the apparatus comprising:
a blood vessel model, which mimics the blood vessel;
a driving device, which moves the blood vessel model in a manner mimicking pulsation of the blood vessel; and
a controller, which controls a manner in which the driving device is driven,
wherein the driving device includes an electroactive polymer actuator.

5. The operation training apparatus according to claim 4, further comprising a base to which the blood vessel model is fixed,
wherein the electroactive polymer actuator is shaped like a sheet and fixed to the base.

6. The operation training apparatus according to claim 1, wherein
the electroactive polymer actuator is formed like a soft sheet, and
the model body is formed like a soft sheet and supported in a state stacked on the electroactive polymer actuator.

7. The operation training apparatus according to claim 6, further comprising a frame member,
wherein a peripheral edge section of the electroactive polymer actuator is supported by the frame member.

8. The operation training apparatus according to claim 6, wherein the model body is supported in a state in which the whole model body is stacked on the electroactive polymer actuator.

9. The operation training apparatus according to claim 6, wherein
the electroactive polymer actuator is a dielectric actuator that includes
a plurality of driving layers arranged on the same plane, and
a common insulating layer for the driving layers, each of the driving layers includes
a dielectric layer that is made of dielectric elastomer, and
a pair of electrode layers that is made of conductive elastomer and sandwiches the dielectric layer,
the insulating layer sandwiches the driving layers from opposite sides in a thickness direction of the driving layers, and
the model body is arranged on the insulating layer in a manner spreading across the driving layers.

10. The operation training apparatus according to claim 1, wherein the electroactive polymer actuator is a dielectric actuator that includes
a dielectric layer that is made of dielectric elastomer, and
a pair of electrode layers that is made of conductive elastomer and sandwiches the dielectric layer.

11. The operation training apparatus according to claim 4, wherein the electroactive polymer actuator is a dielectric actuator that includes
a dielectric layer that is made of dielectric elastomer, and
a pair of electrode layers that is made of conductive elastomer and sandwiches the dielectric layer.

12. The operation training apparatus according to claim 9, wherein the dielectric layer of each driving layer is formed containing a cross-linked polyrotaxane.

13. The operation training apparatus according to claim 10, wherein the dielectric layer is formed containing a cross-linked polyrotaxane.

14. The operation training apparatus according to claim 11, wherein the dielectric layer is formed containing a cross-linked polyrotaxane.

15. An operation training apparatus used in training for operations on a living tissue, comprising:
a model body that mimics the living tissue; and
a soft membrane that supports the model body.

16. The operation training apparatus according to claim 15, wherein the model body is formed like a soft sheet and supported in a state stacked on the soft membrane.

17. The operation training apparatus according to claim 15, wherein the model body is supported in a state in which the whole model body is stacked on the soft membrane.

18. The operation training apparatus according to claim 15, wherein the soft membrane has such an elastic property that, when a load of 10 g is applied to an area that is 20 mm in diameter in which the model body is arranged on the soft membrane, the area is displaced downward by an amount of 1 to 100 mm.

19. The operation training apparatus according to claim 15, further comprising a driving device that moves the model body in a manner mimicking expansion-contraction of the living tissue,
wherein the driving device includes the soft membrane.

20. The operation training apparatus according to claim 19, wherein the soft membrane is an electroactive polymer actuator.

21. The operation training apparatus according to claim 20, wherein
the electroactive polymer actuator is a dielectric actuator, and
the soft membrane includes
a dielectric layer that is made of dielectric elastomer, and
a pair of electrode layers that is made of conductive elastomer and sandwiches the dielectric layer.

22. The operation training apparatus according to claim 21, wherein
the soft membrane includes
a plurality of driving layers arranged on the same plane, and
a common insulating layer for the driving layers,
the dielectric layer is one of a plurality of dielectric layers each arranged in one of the driving layers,
the pair of electrode layers is one of a plurality of pairs of dielectric layers each arranged in one of the driving layers,
the insulating layer sandwiches the driving layers from opposite sides in a thickness direction of the driving layers, and
the model body is arranged on the insulating layer in a manner spreading across the driving layers.
